(19) United States
(12) Patent Application Publication
(10) Pub. No.: US 2023/0027972 A1
(43) Pub. Date: Jan. 26, 2023

(54) ORTHOPEDIC INSOLE

(71) Applicant: Li-Da Huang, Austin, TX (US)

(72) Inventor: Li-Da Huang, Austin, TX (US)

(21) Appl. No.: 17/958,323

(22) Filed: Oct. 1, 2022

Related U.S. Application Data
(63) Continuation of application No. 15/298,153, filed on Oct. 19, 2016, now abandoned.

(51) Int. Cl. A43B 7/1445; A43B 17/02; A61B 5/103; A61B 5/00; B29D 35/00; B29D 35/12; B33Y 80/00

(52) U.S. Cl. CPC A43B 7/1445; A43B 17/02; A61B 5/1038; A61B 5/6829; B29D 35/0009; B29D 35/122; B33Y 80/00; A43B 17/006

(57) ABSTRACT

An orthopedic insole may include at least one strength layer and at least one shock absorbing layer. In one embodiment, the strength layer may be relatively rigid and includes a heel portion and an arch portion, contoured to fit the plantar or bottom surface of the foot to provide arch support. The shock absorbing layer may include a plurality of shock absorbing cells such as recoverable honeycombs or any other negative stiffness structure with the capability to recover. A gait analysis that may include an individual's weight transfer trajectory may have to be conducted to determine the structure of the shock absorbing layer. The orthopedic insole may further include an adjusting layer to supplement the strength layer and the shock absorbing layer to make adjustment to the orthopedic insole if needed.

### FIELD OF THE INVENTION

The present invention relates to an orthopedic insole, and more particularly to a customized orthopedic insole having an optimal and dynamic suspension layers to provide better support for the users.

### BACKGROUND OF THE INVENTION

Currently, there are various mechanisms to offer various degrees of support and control of the foot. Insoles such as arch supports, customized footbed and orthotics have been known for years, and have generally been provided for shoes and other articles for footwear to reduce the shock force created when impact between the heel of the human body and a ground surface result in a reaction force when walking, running or other activities. Furthermore, insoles have been provided to add comfort and support when the body stands motionless by containing a variety of flexible or inflexible materials capable of being contoured to the shape of the underside of an individual's foot.

However, most insoles cannot provide adequate arch support because the arch height of the users varies. Also, most insoles are made with a lower arch height because the insoles with high arch height may create excessive pressure under the arch and the user may feel uncomfortable. Thus, some orthopedic insoles are developed to accommodate various sizes and needs of the users and are customizable according to the specific needs of the foot.

Custom-made orthotics are generally formed from hard plastics by using a mold or extensive measurements of an individual's foot, and modified as needed to provide prescribed corrections by a podiatrist. Unfortunately, most customized shoe inserts require molding of the foot and fabrication of the device with a delay of several weeks between the taking of measurements for an orthotic insert or insole and the arrival of the new customized shoe inserts or insoles. Also, most measurements of the individual's foot do not take the individual's gait into consideration so the individual may still feel uncomfortable during ambulation. Therefore, there remains a need for a new and improved orthopedic insole to overcome the problems stated above.

### SUMMARY OF THE INVENTION

It is an object of the present invention to provide an orthopedic insole that can be customized to according to the needs to each individual's foot.

It is another object of the present invention to provide an orthopedic insole that is made according to the anatomical structure of the individual's foot and gait analysis.

It is a further object of the present invention to provide a customized orthopedic insole that can be designed to meet the healing requirements for pathological conditions, enhance the performance of sports players, and prevent injuries for old people during aging process.

In one aspect, an orthopedic insole may include at least one strength layer and at least one shock absorbing layer. In one embodiment, the strength layer may be relatively rigid and includes a heel portion and an arch portion, contoured to fit the plantar or bottom surface of the foot to provide arch support. The contour of an individual's foot can be obtained by disposing the foot on a formable soft material, such as a foam, and the strength layer can be made according to the contour of a soft material. In one embodiment, the strength layer is made of a thermoformable material, and can be manufactured through an injection molding process. In another embodiment, the soft material can be electrically connected to an electronic device to obtain the individual's foot contour electronically, and the strength layer can be made through a 3D printing process.

It is noted that even though the strength layer is relatively rigid as stated above, when an external force is applied to the strength layer, the strength layer is still configured to change the shape thereof to respond to the external force but will return to its original shape.

The shock absorbing layer may include a plurality of shock absorbing cells such as recoverable honeycombs or any other negative stiffness structure with the capability to recover. The structure and density of these shock absorbing cells are designed to have different thresholds to fit the specific needs of the individuals.

To determine the structure and density, namely stiffness, of the shock absorbing cells, one or more times of “test walk” may have to be conducted on the individual. During ambulation, the individual's gait can be recorded and analyzed. The gait analysis may include an individual's weight transfer trajectory may have to be conducted to determine the stiffness of the shock absorbing layer. The orthopedic insole may further include adjusting layer to supplement the strength layer and the shock absorbing layer to make adjustment to the orthopedic insole if needed.

### BRIEF DESCRIPTION OF THE DRAWINGS

FIG. 1 illustrates a schematic view of the orthopedic insole in the present invention.

FIG. 2 illustrates one embodiment to obtain the individual's foot contour in the present invention.

FIG. 3 illustrates a schematic view of the shock absorbing cell in the shock absorbing layer in the present invention.

FIG. 4 illustrates a schematic view of how to determine the structure and density of the shock absorbing cells in the shock absorbing layer in the present invention.

FIG. 5 illustrates a schematic view of a gait analysis including a plurality of weight transfer trajectories.

### DETAILED DESCRIPTION OF THE INVENTION

The detailed description set forth below is intended as a description of the presently exemplary device provided in accordance with aspects of the present invention and is not intended to represent the only forms in which the present invention may be prepared or utilized. It is to be understood, rather, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described can be used in the practice or testing of the invention, the exemplary methods, devices and materials are now described.

All publications mentioned are incorporated by reference for the purpose of describing and disclosing, for example, the designs and methodologies that are described in the publications that might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

As used in the description herein and throughout the claims that follow, the meaning of “a”, “an”, and “the” includes reference to the plural unless the context clearly dictates otherwise. Also, as used in the description herein and throughout the claims that follow, the terms “comprise or comprising”, “include or including”, “have or having”, “contain or containing” and the like are to be understood to be open-ended, i.e., to mean including but not limited to. As used in the description herein and throughout the claims that follow, the meaning of “in” includes “in” and “on” unless the context clearly dictates otherwise.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the embodiments. As used herein, the term “and/or” includes any and all combinations of one or more of the associated listed items.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

In order to further understand the goal, characteristics and effect of the present invention, a number of embodiments along with the drawings are illustrated as following:

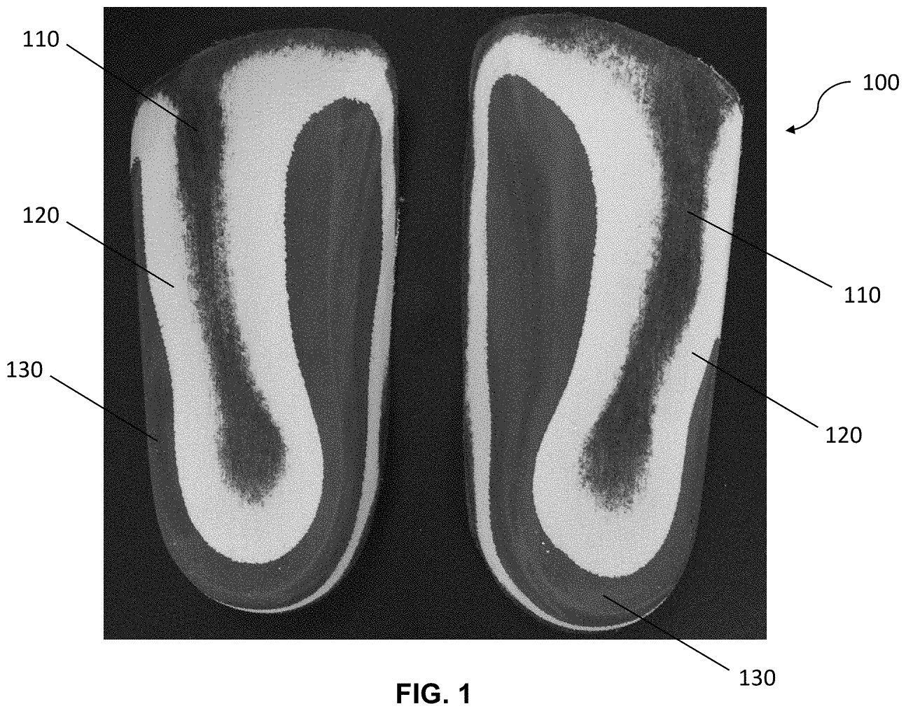

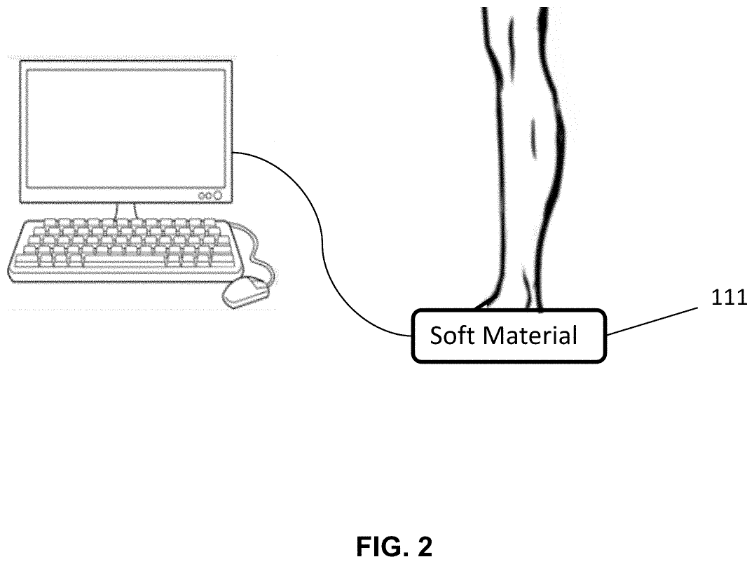

In one aspect, referring to FIG. 1, an orthopedic insole 100 may include at least one strength layer 110 and at least one shock absorbing layer 120. In one embodiment, the strength layer 110 may be relatively rigid and includes a heel portion and an arch portion, contoured to fit the plantar or bottom surface of the foot to provide arch support. The contour of an individual's foot can be obtained by disposing the foot on a formable soft material, such as a foam, and the strength layer 110 can be made according to the contour of a soft material 111. In one embodiment, the strength layer 110 is made of a thermoformable material, and can be manufactured through an injection molding process. In another embodiment, the strength layer 110 can be made through a vacuum pressing process, in which the material of the strength layer 110 is heated up to a predetermined temperature, put on top of a cast, and pressed under a vacuum condition to form the 3D contour. In a further embodiment, the soft material 111 can be electrically connected to an electronic device to obtain the individual's foot contour as shown in FIG. 2, and the strength layer 110 can be made through a 3D printing process.

It is noted that even though the strength layer 110 is relatively rigid as stated above, when an external force is applied to the strength layer 110, the strength layer 110 is still configured to change the shape thereof to respond to the external force but will return to its original shape.

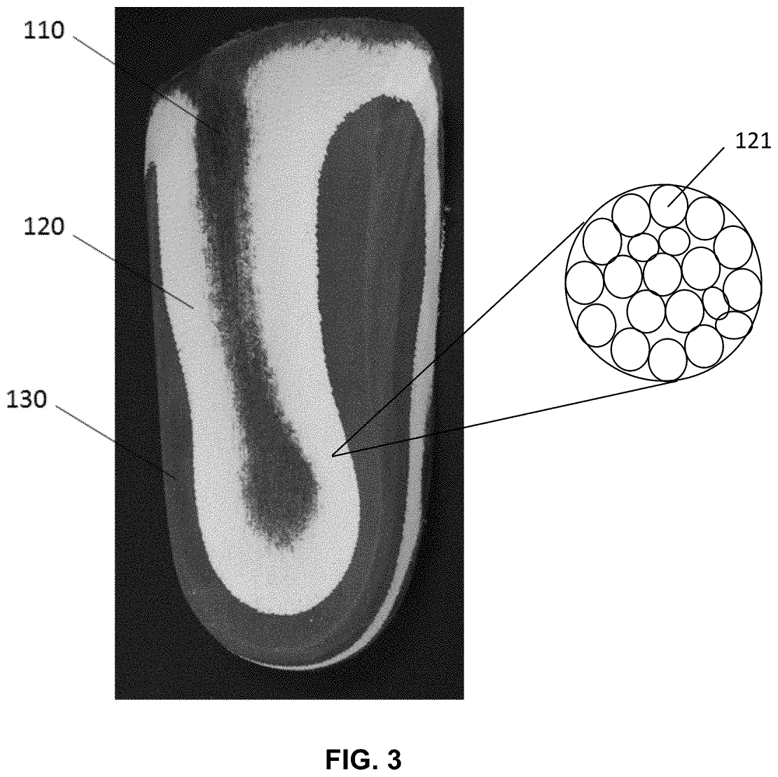

As shown in FIG. 3, the shock absorbing layer 120 may include a plurality of shock absorbing cells 121. In one embodiment, the shock absorbing cells 121 are recoverable honeycombs. In another embodiment, the shock absorbing cells 121 can be a negative stiffness structure with the capability to recover. Generally speaking, when a same amount of external force is applied to cause the shape change, the negative stiffness structure has a shorter responsive time to return to its original shape than the honeycombs. However, the honeycombs may have better shock absorbing capability than the negative stiffness structure. The structure of these shock absorbing cells 121 are designed to have different thresholds to fit the specific needs of the individuals by choosing either honeycombs, negative stiffness structure, or a combination of both.

As shown in FIG. 3, the shock absorbing layer 120 may include a plurality of shock absorbing cells 121. In one embodiment, the shock absorbing cells 121 are foams with cell structures. The cell structure could be open-cell, close-cell, or honeycomb cell structure to absorb the shocking waves. In another embodiment, the shock absorbing cells 121 can be a negative stiffness structure with the capability to recover. Generally speaking, when an external force is applied to cause the shape change, the negative stiffness structure has a shorter responsive time to return to its original shape than the regular honeycombs structure. The strength layer 110, which will keep its molded shape when the external force like shock wave has been removed, and speed up the recovery time of the negative stiffness structured layer. The design of the structures to have the good shock absorbing with adequate recovery time would be a choice based on the needs and the ambulation gait. The structure of these shock absorbing cells 121 are designed to have different thresholds to fit the specific needs of the individuals by choosing either honeycombs, negative stiffness structure, or a combination of both.

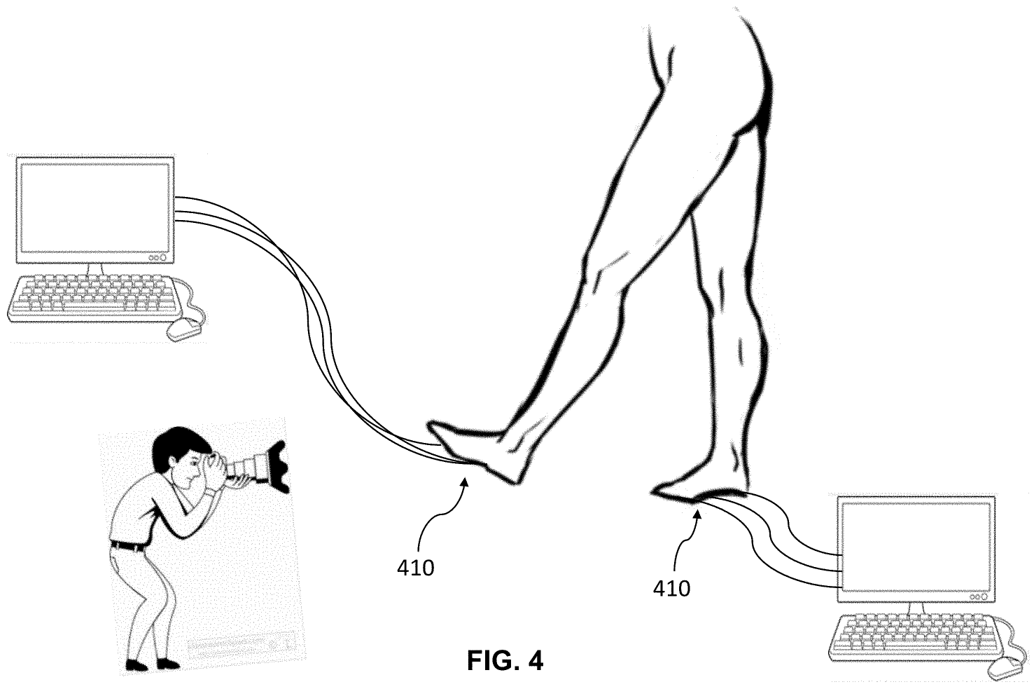

To determine the structure of the shock absorbing cells 121, one or more times of “test walk” may have to be conducted on the individual. During ambulation, the individual's gait can be recorded and analyzed. In one embodiment, the gait analysis can be conducted by a podiatrist by merely watching the individual's walking style. In another embodiment, the gait analysis can be done through an electrical analysis by connecting a plurality of sensors 410 on the individual's foot as shown in FIG. 4. In a further embodiment, the gait analysis can also be obtained by video processing to observe and analyze the movements associated with the joints and muscles.

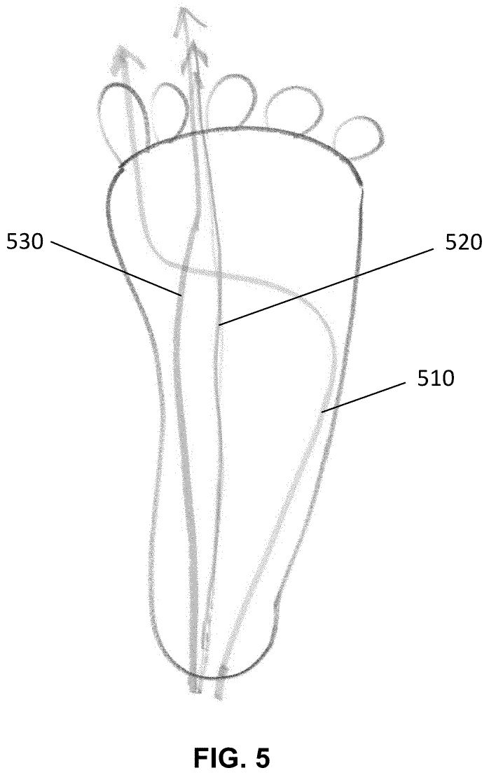

FIG. 5 illustrates a portion of the gait analysis which may include a weight transfer trajectory. More specifically, the weight transfer trajectory (WTT) indicates how a center of mass of an individual travels on the foot during the individual's ambulation. For example, the center of mass may be too close to the lateral side of the foot during ambulation if an individual's weight transfer trajectory is like line 510, while the center of mass may be too close to the medial side of the foot if an individual's weight transfer trajectory is like line 530. In a normal condition, the weight transfer trajectory is close to the center of the foot as shown in line 520. It is noted that the weight transfer trajectory may be obtained by putting a thin film at a plantar surface of the foot inside the shoes, and when the individual walks or runs, the film is configured to record the pressure on the plantar surface during the activities. The film can then be processed to obtain the weight transfer trajectory. In one embodiment, the thin film can be disposed into an athlete's shoes to record the pressure distribution on his/her foot, and based on the pressure distribution thereon, a weight transfer trajectory can be obtained to produce an orthopedic insole according to the present invention to enhance the performance of the athlete. In a further embodiment, a sensing element can be disposed into the orthopedic insole and when the shock wave is beyond a threshold value, the sensing element will trigger a collapse of the shock absorbing layer 120 to provide a much better protection for the individual if the individual happens to suffer from a high shock wave impact such as jumping down from a building.

After obtaining the weight transfer trajectory and observing and analyzing the movements associated with the joints and muscles, the structure of the shock absorbing cells 121 can be determined, and the contour of the strength layer 110 can also be modified according to correct the individual's walking style if needed. If the individual's weight transfer trajectory is like 510, the individual's walking style may be corrected by providing him/her the orthopedic insole with a stiffer shock absorbing layer 120 on the outer portion of the insole, or providing him/her a higher strength layer 110 on the outer portion of the insole. It is noted that the orthopedic insole 100 may further include an adjusting layer 130 to supplement the strength layer and the shock absorbing layer to make adjustment to the orthopedic insole if needed.

Having described the invention by the description and illustrations above, it should be understood that these are exemplary of the invention and are not to be considered as limiting. Accordingly, the invention is not to be considered as limited by the foregoing description, but includes any equivalents.

What is claimed is:

1. A method for manufacturing an orthopedic insole comprising steps of:
generating at least one strength layer of the orthopedic insole; said at least one strength layer having a heel portion and an arch portion, configured to contour and fit a plantar or bottom surface of a foot of an individual to provide arch support;
generating at least one shock absorbing layer of the orthopedic insole, said at least one shock absorbing layer having a plurality of shock absorbing cells; and
conducting a gait analysis to determine a structure of the shock absorbing cells,
wherein the gait analysis includes a plurality of sensors configured to connect on the individual's foot, and a video processing unit configured to observe and analyze the movements associated with the individual's joints and muscles; and a determination of the individual's weight transfer trajectory by disposing a thin film into the individual's shoes to record the pressure distribution, and the structure of the shock absorbing cells is determined after obtaining the individual's weight transfer trajectory and analyzed movements associated with the individual's joints and muscles.

2. The method for manufacturing orthopedic insole of claim 1, wherein the step of generating at least one strength layer of the orthopedic insole includes a step of utilizing a formable soft material to support the individual's foot to obtain a contour of the individual's foot, and the strength layer is made according to the contour of the soft material.

3. The method for manufacturing orthopedic insole of claim 1, wherein the step of generating at least one strength layer of the orthopedic insole includes a step of producing the strength layer of a thermoformable material through an injection molding process.

4. The method for manufacturing orthopedic insole of claim 2, wherein the step of utilizing a formable soft material to support the individual's foot to obtain a contour of the individual's foot includes a step of utilizing an electronic device electrically to connect to the soft material to receive the individual's foot contour, and the strength layer is made through a 3D printing process.

5. The method for manufacturing orthopedic insole of claim 1, wherein the step of generating at least one strength layer of the orthopedic insole includes a step of producing the strength layer through a vacuum pressing process, in which the strength layer is heated up to a predetermined temperature, put on top of a cast, and pressed under a vacuum condition to form the 3D contour.

6. The method for manufacturing orthopedic insole of claim 1, further comprising a step of generating an adjusting layer to supplement the strength layer and the shock absorbing layer to make adjustment to the orthopedic insole.
